(19) United States
(12) Patent Application Publication
(10) Pub. No.: US 2023/0126856 A1
(43) Pub. Date: Apr. 27, 2023

(54) REAGENTS FOR RAPID CHIRAL LABELING AND ANALYSIS OF AMINE CONTAINING ENANTIOMERS

(71) Applicant: Waters Technologies Corporation, Milford, MA (US)

(72) Inventors: Matthew A. Lauber, North Smithfield, RI (US); Darryl W. Brousmiche, Grafton, MA (US); Jun Yang, Franklin, MA (US); Weiqiang Gu, Mansfield, MA (US); Mingcheng Xu, Lexington, MA (US); Peng Chen, Lebanon, CT (US)

(73) Assignee: Waters Technologies Corporation, Milford, MA (US)

(21) Appl. No.: 17/970,962

(22) Filed: Oct. 21, 2022

Related U.S. Application Data
(60) Provisional application No. 63/270,740, filed on Oct. 22, 2021.

(51) Int. Cl. G01N 33/68; C07D 207/46

(52) U.S. Cl. CPC G01N 33/6812; G01N 33/6848; C07D 207/46

(57) ABSTRACT

Provided herein chiral derivatization reagents for use in separating and detecting of amine containing enantiomers. The said chiral derivatization reagents provide a combination of improved detectable properties to facilitate various downstream analyses. In particular, the chiral derivatization reagents include at least one chiral carbon atom; at least one strongly basic moiety; at least one chromophore moiety or at least one fluorophore moiety; and at least one reactive group. The present disclosure further provides methods for analyzing amine-containing enantiomeric isomers using a chromatographic separation device and a mass spectroscopy.

### CROSS-REFERENCE TO RELATED APPLICATION

This application claims priority and benefit to U.S. Provisional Patent Application No. 63/270,740, filed Oct. 22, 2021, entitled “REAGENTS FOR RAPID CHIRAL LABELING AND ANALYSIS OF AMINE CONTAINING ENANTIOMERS”, the contents of which is incorporated herein by reference in its entirety.

### FIELD OF THE TECHNOLOGY

The present disclosure relates generally to chiral derivatization reagents for use in separating and detecting of amine containing enantiomers. The said chiral derivatization reagents provide a combination of improved detectable attributes to facilitate various downstream analyses. In particular, the chiral derivatization reagents of the present disclosure include at least one chiral carbon atom; at least one strongly basic moiety; at least one chromophore moiety or at least one fluorophore moiety; and at least one reactive group. The present disclosure further provides methods for analyzing amine-containing enantiomeric isomers using a chromatographic separation device and a mass spectroscopy.

### BACKGROUND

The separation of enantiomers of biological molecules as well as medicinal drugs is of great importance in biological research and pharmaceutical chemistry because a significant difference in the activity of the enantiomers is generally observed in biological systems. Chiral separations can be carried out by direct resolution that employs chiral stationary-phase (CSP) column containing immobilized chiral selectors or by indirect resolution. The direct method using CSP column is simple and reliable for enantioseparation of chiral molecules. However, the highly sensitive detection in complicated matrices such as plasma and urine is generally difficult. In addition, the CSP columns are comparatively expensive and are sometimes applicable only in normal phase mode, which is not always suitable for biological sample analysis. Furthermore, it can generally be difficult to optimize chiral chromatography to yield resolving power that approaches the peak capacities afforded by reversed phase or hydrophilic interaction chromatography.

The indirect resolution method, on the other hand, is attractive for trace analysis of enantiomers in biological samples such as blood and urine. The indirect resolution of enantiomers involves a derivatization step with a chiral-labeling reagent, e.g. chiral derivatization reagent. A pair of enantiomers is labeled with a chiral derivatization reagent to yield a pair of diastereomers (non-superimposable, non-mirror images) which are subsequently separated by means of standard achiral chromatography. The separation of diastereomers is based on the differences in the physicochemical properties of the diastereomers with an achiral stationary phase.

Notable progress on this pursuit has occurred in the last couple years. Many chiral labeling reagents for ultraviolet-visible and fluorescence detections have been developed for various functional groups, such as amine and carboxylic acid. For example, various optically active labeling reagents for UV-VIS (e.g., 2,3,4,6-tetra-O-acetyl-β-D-glucopyranosyl isothiocyanate (GITC) and Marfey's reagent) and FL (e.g., o-phthalaldehyde (OPA)/chiral thiols and 4-(N,N-dimethylaminosulfonyl)-7-(3-aminopyrrolidin-1-yl)-2,1,3-benzoxadiazole (DB D-APy)) detections have been developed for different functional groups in the chiral molecules.

However, there are hardly any labeling reagents that combine multiple detectable properties to benefit from multiple downstream analysis methods for sensitive and selective determination of enantiomers. In addition, the indirect method which involves a derivatization step of the target molecule by a chiral reagent is time-consuming due to long reaction times needed to perform derivatization reactions.

### SUMMARY

In general, it is an object of the present technology to obviate or mitigate at least one disadvantage of previous chiral derivatization reagents. The chiral derivatization reagents disclosed in the present application is suitable for analysis of amine-containing enantiomers. In some embodiments, amine-containing enantiomers are biologically active molecules, e.g. an amino acid or a biogenic amine.

In one aspect, the chiral derivatization reagents of the present disclosure have one or more chiral centers and one or more chemical properties (e.g., detectable attributes) to facilitate downstream analyses selected from liquid chromatography, ultraviolet-visible (UV-VIS) spectroscopy, fluorescence (FL) spectroscopy, ion mobility, mass spectrometry (MS), or combination thereof. In some embodiments, the detectable attributes are selected from UV-VIS absorptivity, FL quantum yield, high proton affinity or combination thereof. In some embodiments, the high proton affinity is for enhancing MS signaling e.g. MS sensitivity.

In some embodiments, FL quantum yield is between about 30% to about 99%, preferably about 50% to about 99%, more preferably about 80% to about 99%. In some embodiments, the high proton affinity is attributable to a high proton affinitive moiety. In some embodiments, the high proton affinitive moiety has a pKa value greater than 8. In some embodiments, UV-VIS absorptivity has molar absorption coefficient (c) greater than 30 000 M⁻¹ cm⁻¹, greater than 50 000 M⁻¹ cm⁻¹, greater than 70 000 M⁻¹ cm⁻¹, or greater than 80 000 M⁻¹ cm⁻¹.

In some embodiments, the chiral derivatization reagents facilitate liquid chromatography-mass spectroscopy (LC-MS) analysis of amine-containing enantiomers and the said attributes include a highly proton affinitive moiety e.g. strongly basic moiety to enhance MS sensitivity.

In some embodiments, the chiral derivatization reagents facilitate (LC-MS) analysis and (UV-VIS) or (FL) spectroscopy analysis of amine-containing enantiomers, and said attributes include a strongly basic moiety, and high molar UV-VIS absorptivity or a high FL quantum yield.

In another aspect, the chiral derivatization reagents of the present disclosure can rapidly e.g. under 40 minutes undergo a labelling reaction e.g. derivatization reaction with amine-containing enantiomers.

In one aspect, provided herein is a chiral derivatization reagent including a) at least one chiral carbon atom; b) at least one strongly basic moiety having a pKa value greater than 8; c) at least one chromophore moiety or at least one fluorophore moiety, wherein the at least one chromophore moiety or the at least one fluorophore moiety comprises an unsubstituted or substituted aryl or heteroaryl group, which optionally may be condensed with a monocyclic ring or polycyclic ring system and/or may be bonded to a monocyclic ring or polycyclic ring system via a linear or branched alkylene, alkenylene or alkinylene group; and d) at least one reactive group.

In some embodiments, the at least one strongly basic moiety includes at least one of a primary amine group, a secondary amine group, a tertiary amine group, an amidine group, a sulphonic acid ester, a sulfuric ester, a phosphate ester, a phosphonate ester, or combination thereof.

In some embodiments, the at least one reactive group includes at least one of a succinimidyl ester, a succinimidyl carbonate, a succinimidyl carbamate, a sulfosuccinimidyl ester, a sulfosuccinimidyl carbonate, or a sulfosuccinimidyl carbamate.

In another aspect, provided herein is a chiral derivatization reagent including at least one chiral carbon atom, wherein the chiral derivatization reagent has a formula of:

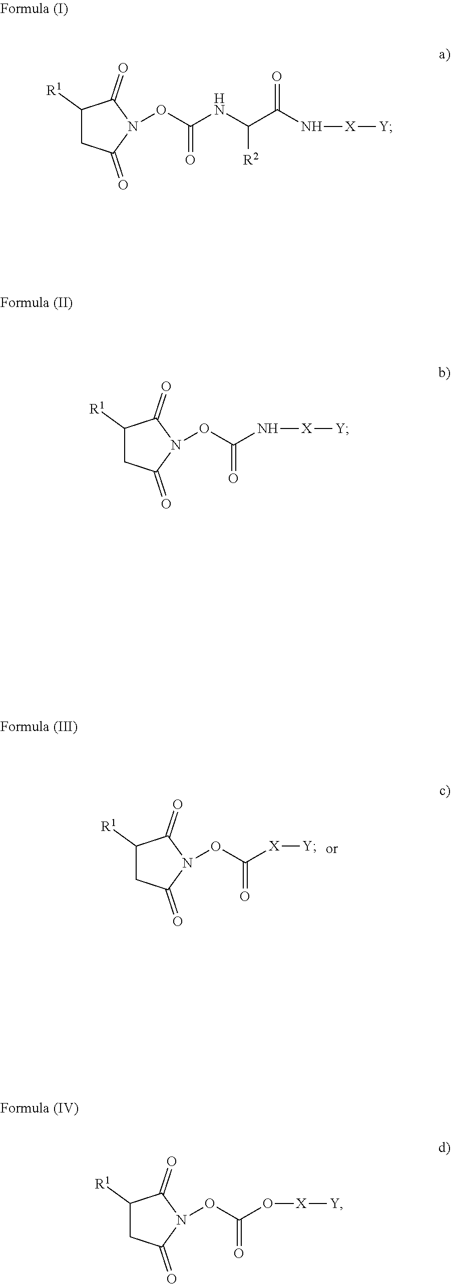

wherein

R¹ is an optional sulfo group, R² is an natural or non-natural amino acid side chain or derivative thereof, X is a linker group comprising linear or branched substituted alkyl chain, linear or branched unsubstituted alkyl chain, linear or branched substituted heteroalkyl chain, or linear or branched unsubstituted heteroalkyl chain, Y is at least one chromophore moiety or at least one fluorophore moiety, wherein Y is optionally functionalized with at least one strongly basic moiety having a pKa value greater than 8.

In some embodiments, the at least one chromophore moiety or the at least one fluorophore moiety includes an unsubstituted or a substituted aryl or heteroaryl group. In some embodiments, the unsubstituted or the substituted aryl or heteroaryl group are condensed with a monocyclic ring. In some embodiments, the unsubstituted or the substituted aryl or heteroaryl group are bonded to a monocyclic ring via a linear or branched alkylene, alkenylene or alkinylene group. In some embodiments, the unsubstituted or the substituted aryl or heteroaryl group are condensed with a polycyclic ring system. In some embodiments, the unsubstituted or the substituted aryl or heteroaryl group are bonded to a polycyclic ring system via a linear or branched alkylene, alkenylene or alkinylene group.

In some embodiments, the chiral derivatization reagent having Formula (I) has the structure of:

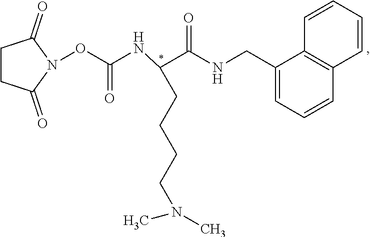 [SMILES: CN(C)CCCCC(NC(=O)ON1C(=O)CCC1=O)C(=O)NCc1cccc2ccccc12]

wherein the carbon atom marked with an asterisk is the at least one chiral carbon atom.

In some embodiments, the chiral derivatization reagent having Formula (I) has the structure of:

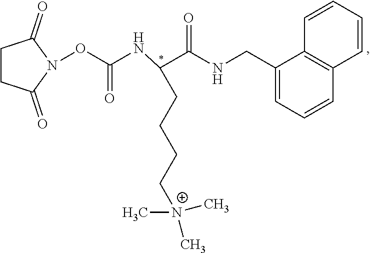 [SMILES: C[N+](C)(C)CCCCC(NC(=O)ON1C(=O)CCC1=O)C(=O)NCc1cccc2ccccc12]

wherein the carbon atom marked with an asterisk is the at least one chiral carbon atom.

In some embodiments, the chiral derivatization reagent having Formula (II) has a structure selected from:

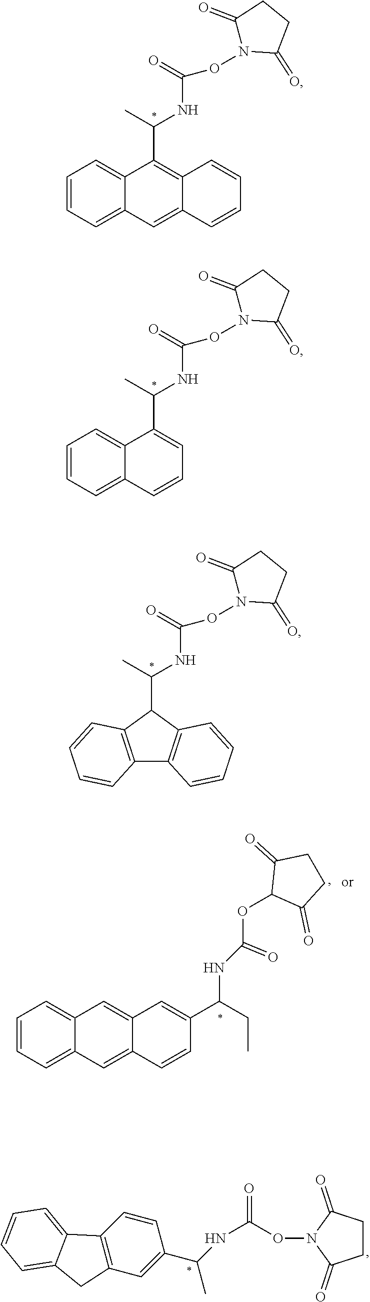 [SMILES: CC(NC(=O)ON1C(=O)CCC1=O)c1c2ccccc2cc2ccccc12 ; CC(NC(=O)ON1C(=O)CCC1=O)c1cccc2ccccc12 ; CC(NC(=O)ON1C(=O)CCC1=O)C1c2ccccc2-c2ccccc21 ; CCC(NC(=O)OC1C(=O)CCC1=O)c1ccc2cc3ccccc3cc2c1 ; CC(NC(=O)ON1C(=O)CCC1=O)c1ccc2c(c1)Cc1ccccc1-2]

wherein the carbon atom marked with an asterisk is the at least one chiral carbon atom.

In some embodiments, the chiral derivatization reagent having Formula (III) has the structure of:

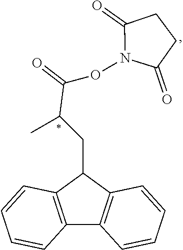 [SMILES: CC(CC1c2ccccc2-c2ccccc21)C(=O)ON1C(=O)CCC1=O]

wherein the carbon atom marked with an asterisk is me at least one chiral carbon atom.

In any of the above aspects and embodiments, the mass of the chiral derivatization reagent has a molecular weight of about 150 to about 600 atomic mass units.

In any of the above aspects and embodiments, the chiral derivatization reagents are for separating amino group-containing enantiomeric isomers in a sample.

In one aspect, provided herein is a method of separating amino group-containing enantiomeric isomers, including using the chiral derivatization reagent of any one above aspects and embodiments.

In another aspect, provided herein is a method for analyzing an amino group-containing enantiomeric isomers in a sample including: a) reacting the sample including the amino group-containing enantiomeric isomers with a chiral derivatization reagent of any one above aspects and embodiments; b) allowing each amino group-containing enantiomeric isomers to react with the chiral derivatization reagent of any one above aspects and embodiments to produce a mixture of diastereomers comprising the derivatization reagent and a specific amino group-containing enantiomeric isomer; c) loading the mixture of diastereomers onto a chromatographic separation device; d) eluting the mixture of diastereomers from the chromatographic separation device; and analyzing the eluent for the presence of a specific diasteromer including the specific amino group-containing enantiomeric isomer using a mass spectroscopy.

In some embodiments, the chromatographic separation device is a device selected from the group consisting of a chromatographic column, a thin layer plate, a filtration membrane, a microfluidic separation device, a sample cleanup device, a solid support, a solid phase extraction device, a microchip separation device, and a microtiter plate. In some embodiments, the chromatographic separation device is a liquid chromatography column including an achiral stationary phase. In some embodiments, the liquid chromatography column is selected from a reversed phase column, a cation exchange column, an anion exchange column, an ion pair separation column, normal phase column, an ion mobility separation column, a size-exclusion column, a polar nonionic column, or any combination thereof. In some embodiments, the chromatographic separation device is equipped with an optical detector. In some embodiments, the chromatographic separation device is equipped with an optical resolution column.

In some embodiments, the mass spectroscopy is real-time online mass spectroscopy. In some embodiments, the mass spectrometry is selected from the group consisting of matrix-assisted laser desorption/ionization-time-of-flight (MALDI-TOF), electrospray-ionization (ESI), charge detection (CD), fourier transform-ion cyclotron resonance (FT-ICR), ion-mobility spectrometry (IMS), triple quadrupole, time of flight (TOF), and ion trap.

In some embodiments, the sample is a biological sample includes a bodily fluid. In some embodiments, the bodily fluid is selected from the group consisting of saliva, sweat, urine, blood, serum, plasma, spinal fluid, and combinations thereof. In some embodiments, the bodily fluid includes neurotransmitters. In some embodiments, the amino group-containing enantiomer is an amino acid or a biogenic amine.

In one aspect provided herein is a chiral derivatization reagent including at least one chiral carbon atom, wherein the chiral derivatization reagent has a formula of:

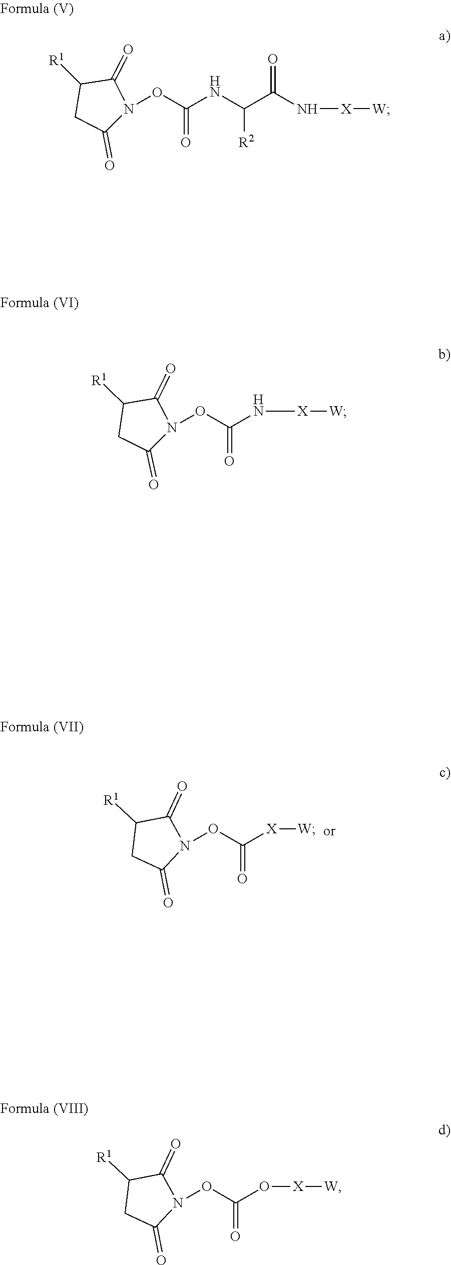

wherein R1 is an optional sulfo group, R2 is an natural or non-natural amino acid side chain or derivative thereof, X is a linker group comprising linear or branched substituted alkyl chain, linear or branched unsubstituted alkyl chain, linear or branched substituted heteroalkyl chain, or linear or branched unsubstituted heteroalkyl chain, W is —[Y—Z] or —[Z—Y], wherein—is the attachment point to X, Y is at least one chromophore moiety or at least one fluorophore moiety, and Z is strongly basic moiety having a pKa value greater than 8.

In some embodiments, the chiral derivatization reagent having Formula (VI) has a structure selected from:

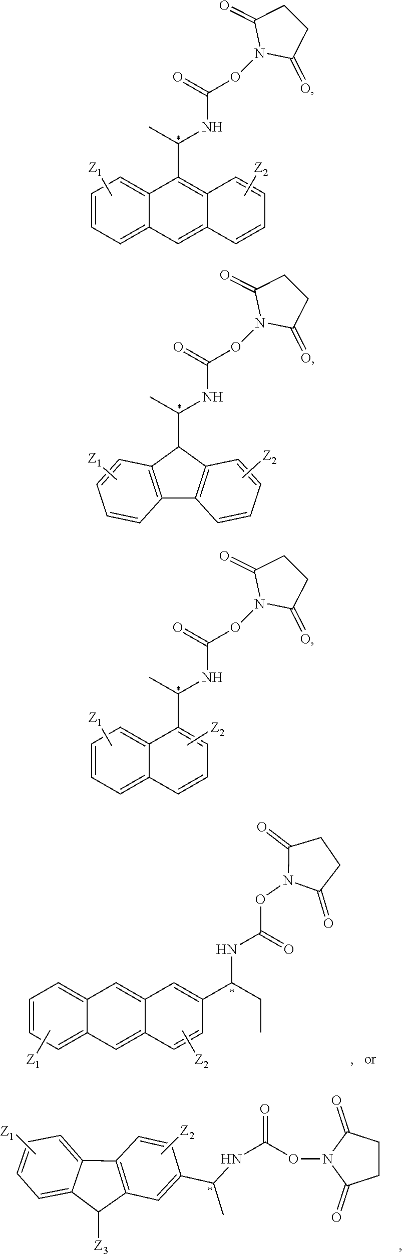

wherein the carbon atom marked with an asterisk is the at least one chiral carbon atom, wherein Z₁, Z₂ and Z₃ are independently hydrogen, or strongly basic moiety having a pKa value greater than 8, and Z₁, Z₂ and Z₃ may be the same or different.

In some embodiments, the chiral derivatization reagent having Formula (VII) has a structure selected from:

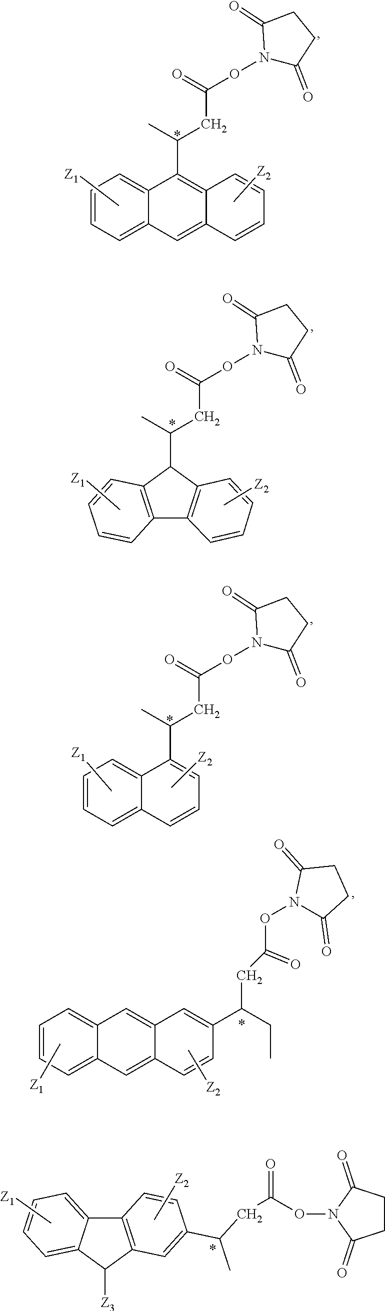

wherein the carbon atom marked with an asterisk is the at least one chiral carbon atom, wherein Z₁, Z₂ and Z₃ are independently hydrogen, or strongly basic moiety having a pKa value greater than 8, and Z₁, Z₂ and Z₃ may be the same or different.

In some embodiments, the chiral derivatization reagent having Formula (VIII) has a structure selected from:

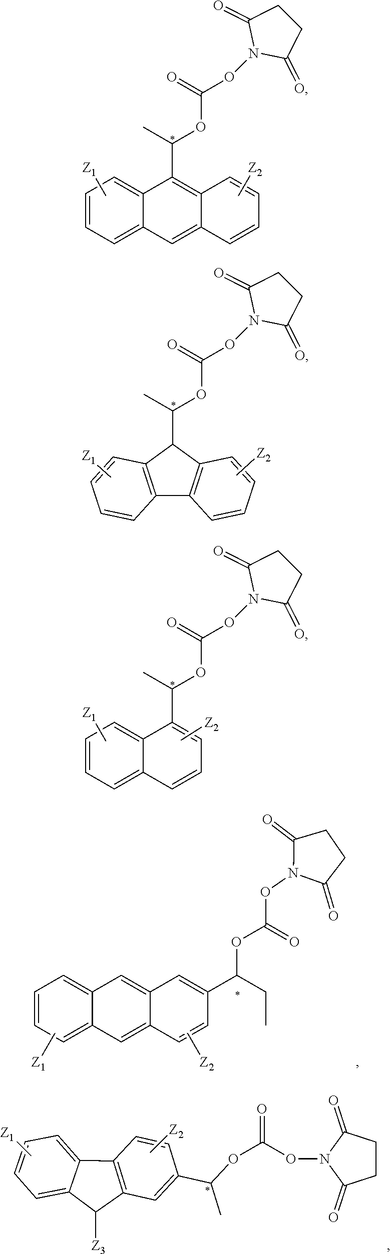

wherein the carbon atom marked with an asterisk is the at least one chiral carbon atom, wherein Z₁, Z₂ and Z₃ are independently hydrogen, or strongly basic moiety having a pKa value greater than 8, and Z₁, Z₂ and Z₃ may be the same or different.

In some embodiments, the strongly basic moiety includes at least one of a primary amine group, a secondary amine group, a tertiary amine group, a amidine group, a sulphonic acid ester, a sulfuric ester, a phosphate ester, a phosphonate ester, or combination thereof.

The chiral derivatization reagents of the present disclosure can advantageously yield highly sensitive and selective detection of amine-containing enantiomers in various matrices. The said chiral derivatization reagents can include multiple attributes to facilitate multiple downstream analysis including liquid chromatography (LC), ultraviolet-visible (UV-VIS) spectroscopy, fluorescence (FL) spectroscopy, ion mobility, mass spectrometry (MS), or combination thereof. For example, said attributes can enhance not only sensitivity of MS, but also resolution of LC. The said chiral derivatization reagents include further attributes to facilitate additional methods combined with LC-MS such as UV-VIS or FL spectroscopy. In some embodiments, the further attributes include at least one chromophore moiety or the at least one fluorophore moiety.

In addition, the chiral derivatization reagents of the present disclosure are fast-reactive molecules that can rapidly undergo a labelling reaction e.g., a derivatization reaction with amine-containing enantiomers. In some embodiments, the derivatization reaction time is under 60 minutes, preferably under 40 minutes, most preferably under 20 minutes.

### BRIEF DESCRIPTION OF THE DRAWINGS

The technology will be more fully understood from the following detailed description taken in conjunction with the accompanying drawings, in which:

FIG. 1 shows mechanism of derivatization reaction of compound F with alanine isomers.

FIG. 2 displays UV chromatograms (A254) of compound F labeled L-alanine, D-alanine and a 50:50 mixture thereof that demonstrate L and D-alanine derivatives can be efficiently separated and baseline resolved. Separations were performed with a 2.1×100 mm 1.8 μm 100 Å C18 column, 0.1% DFA (v/v) modified water and acetonitrile mobile phases, a flow rate of 0.4 mL/min, a column temperature of 60° C., and a 20 min gradient from 2 to 60% acetonitrile. Solutions of 0.5 mM alanine modified with 5 mM reagent (in 50:50 DMF/borate buffer) were injected at a 0.1 μL volume, which corresponds to a per analysis quantity of alanine of approximately 50 pmoles. Peak identifications were facilitated by serial MS analysis.

FIG. 3 displays UV chromatograms (A254) of AccQFluor labeled L-alanine, D-alanine and a 50:50 mixture thereof wherein it is demonstrated that L and D-alanine derivatives co-elute. Separations were performed with a 2.1×100 mm 1.8 μm 100 Å C18 column, 0.1% DFA (v/v) modified water and acetonitrile mobile phases, a flow rate of 0.4 mL/min, a column temperature of 60° C., and a 20 min gradient from 2 to 60% acetonitrile. Solutions of 0.5 mM alanine modified with 5 mM reagent (in 50:50 DMF/borate buffer) were injected at a 0.1 μL volume, which corresponds to a per analysis quantity of alanine of approximately 50 pmoles.

FIG. 4A and FIG. 4B display extracted ion chromatograms of alanine resulting from a 50:50 D and L isomer mixture labeled with AccQFluor (FIG. 4A) versus Compound F (FIG. 4B). Ion signal for the monoisotopic mass of the derivatives were extracted with a 0.1 m/z mass window. Separations were performed with a 2.1×100 mm 1.8 μm 100 Å C18 column, 0.1% DFA (v/v) modified water and acetonitrile mobile phases, a flow rate of 0.4 mL/min, a column temperature of 60° C., and a 20 min gradient from 2 to 60% acetonitrile. Solutions of 0.5 mM alanine modified with 5 mM reagent (in 50:50 DMF/borate buffer) were injected at a 0.1 μL volume, which corresponds to a per analysis quantity of alanine of approximately 50 pmoles.

FIG. 5 displays reaction scheme for the synthesis of MS sensitive chiral derivatization reagent that has a strongly basic moiety.

### DETAILED DESCRIPTION

The determination of enantiomers of biological molecules is an important issue because a significant difference in the activity of the enantiomers is generally observed in biological systems. Sparingly few options exist for the selective detection and quantitation of enantiomeric biological molecules. Mass spectrometry, spectroscopy, and NMR are not of any diagnostic value on their own. Traditionally, chiral chromatography is performed to achieve separations of enantiomers. Enantiomers cannot be effectively separated by achiral stationary phases, but adsorptive chromatography can be performed with stationary phases that themselves exhibit chiral moieties so as to tease apart enantiomeric analytes. Generally, it can be difficult to optimize chiral chromatography to yield resolving power that approaches the peak capacities afforded by reversed phase or hydrophilic interaction chromatography. There is significant interest, as a result, to have methods for separating and/or detecting enantiomeric analytes selectively and sensitively that take advantage of efficient separation and detection techniques such as high-performance liquid achiral chromatography and potentially even ion mobility spectrometry and electrochromatography.

It is known that diastereomers (non-superimposable, non-mirror images) can be separated by means of standard achiral chromatography. This is made possible by the conformation and spatial differences that exist with the combination of one or more chiral centers in the same molecule. Accordingly, it is feasible to derivatize enantiomeric analytes that have one chiral center with an enantiomerically pure labeling reagent e.g., a chiral derivatization reagent, that itself has one or more chiral centers to produce diastereomers derivatives that can be separated and thereby detected or quantified. There are different means of performing derivatization reactions, but in these cases of analyzing enantiomers the common aim is to reliably create diastereomers.

Various chiral derivatization reagent has been synthesized in the last couple years. In 2015, Toyo'oka presented work on two triazine-based labeling reagents, one of which contained an NHS ester and was designed for the derivatization of primary and secondary amines (Toyo'oka et al. Anal Chim Acta 2015, 875, 73-82; Toyo'oka et al. Anal Chim Acta 2015, 898, 73-84). While this label contains 4 nitrogen atoms, their electrons are conjugated together such that there is little to no basicity. In 2020, Harada, Shimbo and co-workers published a report on the development and application of a nitrophenyl activated tag built on a chiral biaryl moiety bearing a weak dimethyl (pKa 4 to 6) aniline functionality. Despite of this, their analyses showed the potential to separate derivatized D and L-amino acids by RPLC and detect them by ESI-MS (Shimbo et al. Symmetry 2020, 12 (913)).

Therefore, the technology of the present disclosure provides chiral derivatization reagents comprising one or more chiral centers and one or more detectable attributes such as highly basic moieties, chromophore moieties or fluorophore moieties to facilitate downstream analyses selected from liquid chromatography, ultraviolet-visible (UV-VIS) spectroscopy, fluorescence (FL) spectroscopy, ion mobility, mass spectrometry (MS), or combination thereof. In some embodiments, the detectable attributes are selected from high molar UV-VIS absorptivity, a high FL quantum yield, high proton affinitive moiety that can enhance MS sensitivity, or combination thereof.

In some embodiments, the chiral derivatization reagents facilitate liquid chromatography-mass spectroscopy (LC-MS) analysis of amine-containing enantiomers and the said attributes include a highly proton affinitive moiety e.g. strongly basic moiety to enhance MS sensitivity.

In some embodiments, the chiral derivatization reagents facilitate liquid chromatography-mass spectroscopy (LC-MS) analysis of amine-containing enantiomers and the said attributes include a highly proton affinitive moiety e.g. strongly basic moiety to enhance MS sensitivity.

In one aspect, provided herein is a chiral derivatization reagent including a) at least one chiral carbon atom; b) at least one strongly basic moiety having a pKa value greater than 8; c) at least one chromophore moiety or at least one fluorophore moiety, wherein the at least one chromophore moiety or the at least one fluorophore moiety comprises an unsubstituted or substituted aryl or heteroaryl group, which optionally may be condensed with a monocyclic ring or polycyclic ring system and/or may be bonded via a linear or branched alkylene, alkenylene or alkinylene group; and d) at least one reactive group.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “sample” refers to any medium that includes an analyte (e.g. an amine containing enantiomer) to be processed using the methods according to the present disclosure. A sample may be selected from an agricultural sample, an environmental sample, or a biological sample. A biological sample may include, but is not limited to, for example, a clinical specimen (e.g., blood, plasma, serum, sputum, tissue, urine, saliva, sample/fluid from the respiratory tract, etc.). A sample may not be a biological sample, but the sample may comprise a biological mixture.

As used herein, the term “optional” or “optionally” refers to a described event or circumstance may or may not occur, and that the description includes instances where the event or circumstance occurs and instances where the event or circumstance does not. For example, “Y is optionally functionalized with at least one strongly basic moiety” refers to Y that may or may not be functionalized and that the description encompasses both functionalized Y and un functionalized Y.

As used herein, the term “fluorophore moiety” refers to a substance that emits light at a longer wavelength when irradiated with light of a specific wavelength, i.e., emits fluorescence. Fluorophore moieties include organic dyes, organometallic compounds, metal chelates, fluorescent conjugated polymers, quantum dots, or nanoparticles, or combinations of the above. In some embodiments, at least one fluorophore moiety has quantum yield of from about 30% to about 99%, preferably from about 50% to about 99%, more preferably from about 80% to about 99%.

As used herein, the term “chromophore moiety” refers to a compound or chemical group that absorbs light at a particular wavelength, thereby producing a color. The chromophore moieties generally have alternating double bonds or conjugated double bonds. The chromophore moieties include at least one selected from the group consisting of an unsaturated alkyl group, an aromatic group, a heterocyclic ring, and a metal ion. Specifically, the chromophore moieties include at least one selected from the group consisting of a nitroso group, a nitro group, an azo group, a methine group, an amino group, a ketone group, a thiazolyl group, a naphthoquinone group, an indoline group, a stilbene derivative, an indophenol derivative, a diphenylmethane derivative, an anthraquinone derivative, a triarylmethane derivative, a diazine derivative, an indigoid derivative, a xanthene derivative, an oxazine derivative, a phthalocyanine derivative, an acridine derivative, a thiazine derivate, a sulfur atom-containing compound, and a metal ion-containing compound. In some embodiments, at least one chromophore moiety has molar absorption coefficient (c) greater than 30 000 M⁻¹ cm⁻¹, greater than 50 000 M⁻¹ cm⁻¹, greater than 70 000 M⁻¹ cm⁻¹, or greater than 80 000 M⁻¹ cm⁻¹.

As used herein, the term “stereoisomer,” “stereoisomeric form,” and the like are used interchangeably herein to refer to all isomers of individual molecules that differ only in the orientation of their atoms in space. It includes enantiomers and isomers of compounds with more than one chiral center that are not mirror images of one another (“diastereomers”).

As used herein, the term “chiral center” refers to an atom holding a set of ligands in a spatial arrangement, which is not superposable on its mirror image.

As used herein, the term “chiral carbon” refers to a central carbon in which all four functional groups around the carbon are different, and the compound having the chiral carbon does not superimpose a real image and a mirror image.

As used herein, the term “enantiomer” or “enantiomeric” refers to a molecule that is nonsuperimposable on its mirror image and hence optically active where the enantiomer rotates the plane of polarized light in one direction and its mirror image rotates the plane of polarized light in the opposite direction. Enantiomers have identical physical properties e.g. melting points, boiling points, densities, refractive indexes, and any other physical constant one might measure, except as to the direction of rotation of the plane of polarized light.

As described herein, the terms “labeling”, “tagging”, or “derivatizing” are used interchangeably through this specification. A “derivatized molecule refers to a molecule that has been labeled or tagged with one of the derivatization reagents of the present technology.

As used herein, the term “biogenic amine” refers to an amine that is the result of a biological process, e.g., metabolism, etc. Biogenic amines are organic bases with low molecular weight and are synthesized by microbial, vegetable and animal metabolisms.

As used herein, the term “alkyl,” as used herein, alone or in combination, refers to a straight-chain or branched-chain alkyl radical containing from 1 to and including 20, preferably 1 to 10, and more preferably 1 to 6, carbon atoms.

As used herein, the term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms can optionally be oxidized and the nitrogen heteroatom can optionally be quaternized. The heteroatom(s) O, N and S can be placed at any interior position of the heteroalkyl group. Up to two heteroatoms can be consecutive, such as, for example, —CH₂—NH—OCH₃.

As used herein, the term “ring system” refers to a monocyclic, bicyclic or polycyclic hydrocarbon ring system, wherein each ring is either completely saturated or contains one or more units of unsaturation, but where no ring is aromatic. Ring systems may have fused rings that share a common carbon atom.

The term “neurotransmitter” refers to a molecule that is synthesized and/or present in a neuron, and that can be released from a neuron to produce a response in a target. For example, neurotransmitters can be released into the synaptic cleft and received by receptors on a target cell. Neurotransmitters include, but are not limited to, aminoacids such as glutamine, glycine, γ-aminobutyric acid (GABA), D-serine, and aspartate; histamine; serotonin; catecholamines, such as norepinephrine, epinephrine, and dopamine; phenethylamines, such as phenethylamine, N-methylphenethylamine and phenylethanolamine; thyronamines; tryptamines (e.g., melatonin); peptides, such as substance P and somatastatin; acetylcholine; and purines, such as adenosine; as well as their metabolites.

As used herein, the term “reactive group” refers to a functional group that can react with a second functional group (e.g., amine group of amine-containing enantiomers) under relatively mild conditions and without the need for preliminary functionalization of the reactive group. Reactive groups can be selected a variety of from well-known reactive groups available in the art, such as but not limited to e.g., hydroxyl groups, thiols, optionally substituted or activated carboxylic acids, isocyanates, amines, esters, thioesters, aldehyde groups, propionaldehydea, butyraldehyde groups, maleimides, and succinimides, and the like. Further non-limiting examples of reactive group reactions are e.g., Suzuki coupling, Heck coupling, Sonogashira coupling, Wittig reaction, alkyl lithium-mediated condensations, halogenation, SN₂ displacements (for example, N, O, S), ester formation, and amide formation, as well as other reactions and reactive groups that can be used to generate chemical entities, such as those presented herein.

The chiral derivatization reagents described herein can be used to label (tag) amine containing enantiomers. Rapid derivatization can proceed, for example, via nucleophilic attack and substitution. If rapid derivatization reagents are utilized such as the compounds described herein, the reaction can be completed in minutes at room temperature.

### EXAMPLES

The following examples are included to demonstrate preferred embodiments of the technology. However, those of skill in the art should, in light of the present disclosure, appreciate that many changes can be made in the specific embodiments which are disclosed and still obtain a like or similar result without departing from the spirit and scope of the technology.

### Example 1: Synthesis of Compound D

A 0.25-gram quantity of (R)-1-(Anthracen-9-yl)ethan-1-amine was dissolved in 2.5 mL acetonitrile and transferred into a dropping funnel. Into a 50 mL flask, 0.3258 grams of DSC was added along with 11.4 mL of acetonitrile. The (R)-1-(Anthracen-9-yl)ethan-1-amine solution was then added to the DSC solution over a 10-minute period. Once the addition was complete, the solution was stirred under nitrogen for 3 hours. Subsequently, acetonitrile solvent was removed from the reaction using a rotavap. The synthesis of compound D was confirmed by ¹H NMR.

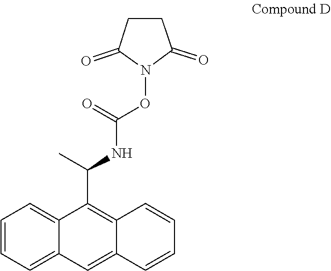 [SMILES: C[C@@H](NC(=O)ON1C(=O)CCC1=O)c1c2ccccc2cc2ccccc12]

### Example 2: Synthesis of Compound F

A 1-gram quantity of (R)-(+)-1-(1-Naphthyl)ethylamine was dissolved in 10 mL acetonitrile and transferred into a dropping funnel. Into a 100 mL flask, 1.65 grams of DSC was added along with 58 mL of acetonitrile. This mixture was stirred for 30 minutes. The (R)-(+)-1-(1-Naphthyl)ethylamine solution was then added to the DSC solution over a 30-minute period. Once the addition was complete, the solution was stirred under nitrogen for 3 hours. Subsequently, acetonitrile solvent was removed from the reaction using a rotavap. The synthesis of compound F was confirmed by ¹H NMR.

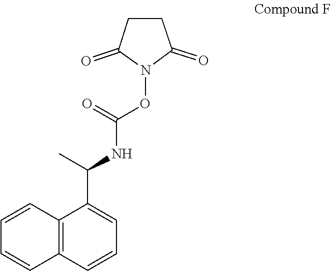 [SMILES: C[C@@H](NC(=O)ON1C(=O)CCC1=O)c1cccc2ccccc12]

### Example 3: Derivatization and Analysis of (D) and (L)-Alanine

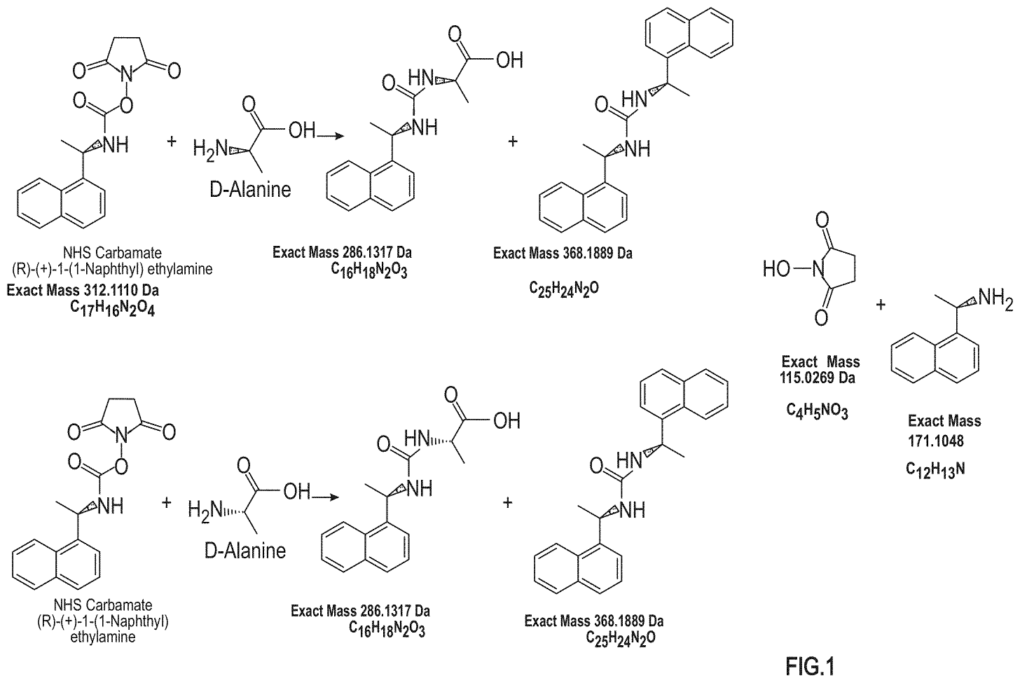

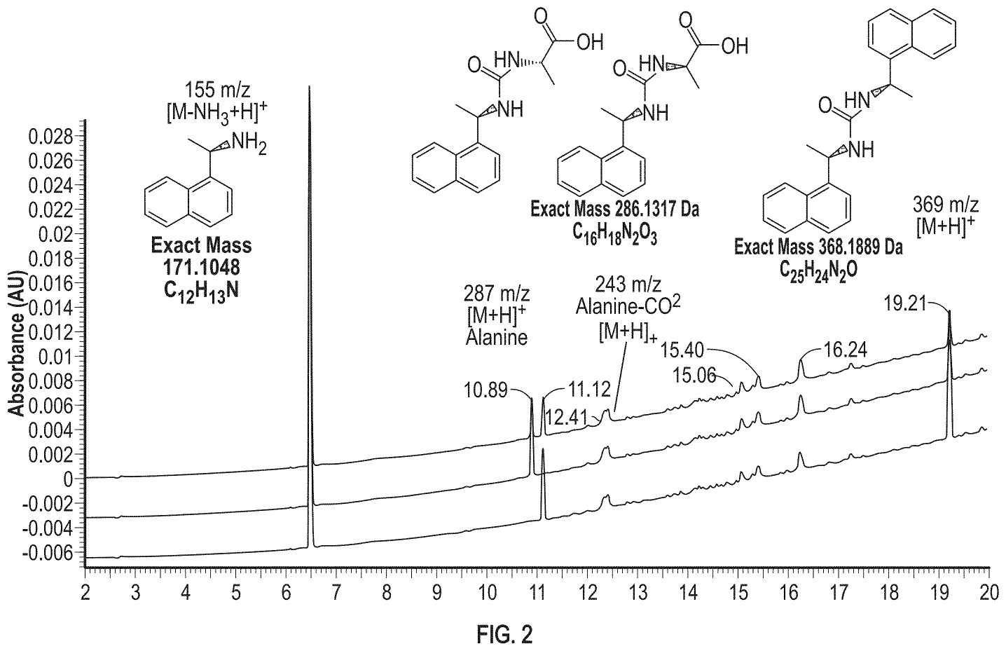

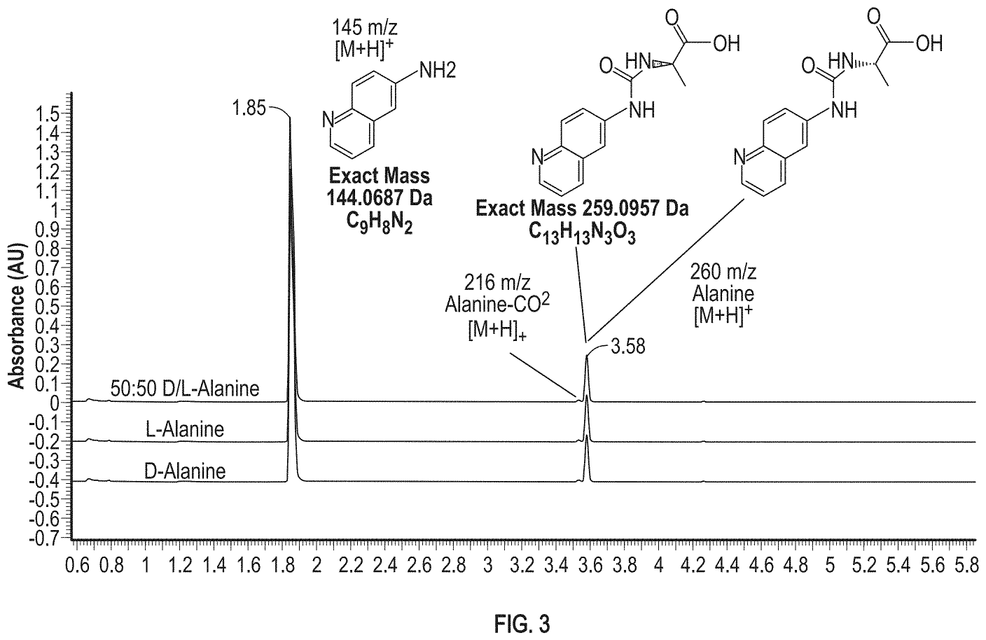

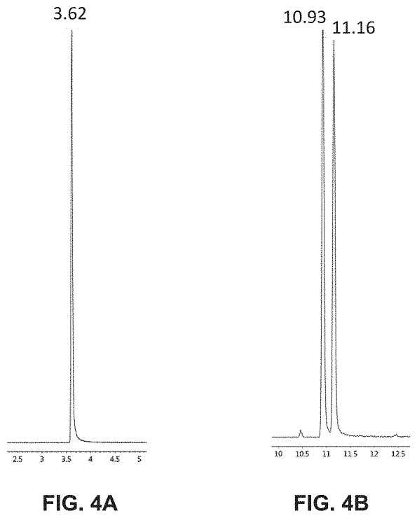

Solutions of 0.5 mM D and L-alanine were derivatized with either 5 mM AccQFluor or 5 mM Compound F in a solvent mixture comprised of 50:50 anhydrous dimethylformamide/200 mM sodium borate in water pH 8.8 (FIG. 1). Reactions were allowed to proceed at room temperature for 10 minutes before being aliquoted for analysis. A solution of 0.25 mM D-alanine combined with 0.25 mM L-alanine was also derivatized and prepared for analysis. Crude reaction mixtures were analyzed by LC-MS with a UHPLC outfitted with a time of flight mass spectrometer. Separations were performed with a 2.1×100 mm 1.7 μm C18 column, 0.1% DFA (v/v) modified water and acetonitrile mobile phases, a flow rate of 0.4 mL/min, a column temperature of 60° C., and a 20 min gradient from 2 to 60% acetonitrile. Solutions of 0.5 mM alanine modified with 5 mM reagent (in 50:50 DMF/borate buffer) were injected at a 0.1 μL volume, which corresponds to a per analysis quantity of alanine of approximately 50 pmoles. Chromatograms resulting from these analyses are provided in FIGS. 2, 3, 4A and 4B. The results show successful separation and detection of D and L-alanine using the derivatization reagents of the present technology.

### Experimental Conditions

| LC: | ACQUITY UPLC | | |
|---|---|---|---|
| Column: | ACQUITY UPLC HSS T3 1.8 μm 100 Å | | |
| | 2.1 × 100 mm column | | |
| Injection Volume: | 0.1 μL | | |
| Column Temperature: | 60° C. | | |
| Flow rate: | 0.4 mL/min | | |
| Mobile phase A: | 0.1% difluoroacetic acid (DFA) in | | |
| | 18.2 MΩ water | | |
| Mobile phase B: | 0.1% difluoroacetic acid (DFA) in acetonitrile | | |
| Gradient: | Time (min) | % A | % B |
| | 0.0 | 98.0 | 2.0 |
| | 20.0 | 40.0 | 60.0 |
| | 21.0 | 10.0 | 90.0 |
| | 22.0 | 98.0 | 2.0 |
| | 30.0 | 98.0 | 2.0 |

| | |
|---|---|
| UV detection: | 190 to 500 nm @ 20 Hz and 1.2 nm |
| | wavelength resolution |
| MS: | Xevo G2-S QTof |
| Acquisition window: | 50 to 1000 m/z |
| Scan rate: | 5 Hz |
| Cone Voltage: | 40 V |

### Example 4: Derivatization and Analysis of (D) and (L)-Alanine

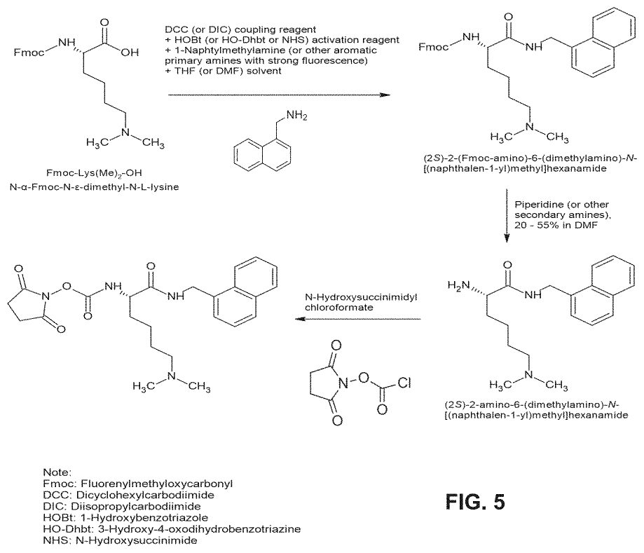

Multiple reaction scheme can be envisioned to manufacture an MS enhancing e.g., MS sensitive chiral derivatization reagent. In one approach, it makes sense to start with an enantiomerically pure amino acid because several are readily available. An example is shown where a chiral Fmoc-Lys(Me)₂-OH is used as the starting material (FIG. 5). The tertiary amine of the lysine side chain can be feasibly protonated during the ESI ionization in positive mode to enhance MS signals. The C-terminal carboxylic acid group can be coupled with a fluorescent or chromogenic aromatic amine by utilizing coupling reactions commonly applied in peptide synthesis. The base-labile Fmoc protected amino group can be deprotected and reacted with N-Hydroxysuccinimidyl chloroformate to form a carbamate derivative and thereby produce the chiral fluorogenic reagent for modification of analytes containing primary and secondary amines. Equivalent reaction schemes can be outlined starting with arginine and homoarginine amino acid building blocks.

While this disclosure has been particularly shown and described with reference to example embodiments thereof, it will be understood by those skilled in the art that various changes in form and details may be made therein without departing from the scope of the technology encompassed by the appended claims. For example, other chromatography systems or detection systems can be used.

1. A chiral derivatization reagent comprising:
a) at least one chiral carbon atom:
b) at least one strongly basic moiety having a pKa value greater than 8;
c) at least one chromophore moiety or at least one fluorophore moiety, wherein the at least one chromophore moiety or the at least one fluorophore moiety comprises an unsubstituted or substituted aryl or heteroaryl group; and
d) at least one reactive group.

2. The chiral derivatization reagent of claim 1, wherein the at least one strongly basic moiety comprises at least one of a primary amine group, a secondary amine group, a tertiary amine group, an amidine group, a sulphonic acid ester, a sulfuric ester, a phosphate ester, a phosphonate ester, or combination thereof.

3. The chiral derivatization reagent of claim 1, wherein the at least one reactive group comprises at least one of a succinimidyl ester, a succinimidyl carbonate, a succinimidyl carbamate, a sulfosuccinimidyl ester, a sulfosuccinimidyl carbonate, or a sulfosuccinimidyl carbamate.

4. A chiral derivatization reagent comprising at least one chiral carbon atom, the chiral derivatization reagent having a formula of: 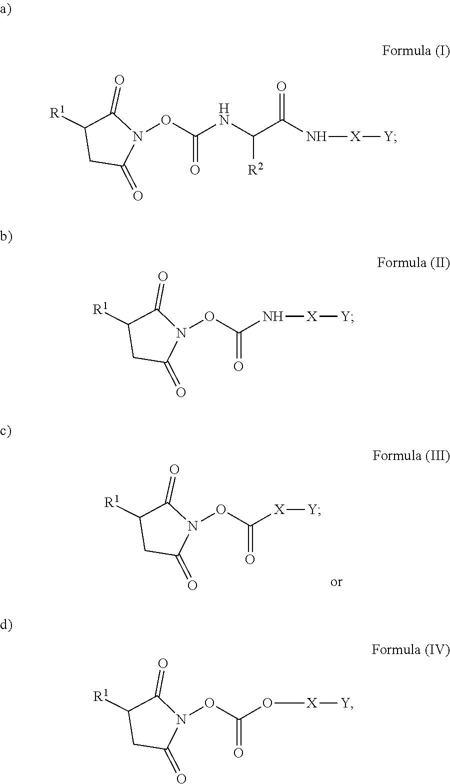
wherein
R¹ is an optional sulfo group, R² is an natural or non-natural amino acid side chain or derivative thereof, X is a linker group comprising linear or branched substituted alkyl chain, linear or branched unsubstituted alkyl chain, linear or branched substituted heteroalkyl chain, or linear or branched unsubstituted heteroalkyl chain, Y is at least one chromophore moiety or at least one fluorophore moiety, wherein Y is optionally functionalized with at least one strongly basic moiety having a pKa value greater than 8.

5. The chiral derivatization reagent of claim 4, wherein the at least one chromophore moiety or the at least one fluorophore moiety comprises an unsubstituted or a substituted aryl or heteroaryl group.

6-9. (canceled)

10. The chiral derivatization reagent of claim 4, wherein the chiral derivatization reagent having Formula (I) has the structure of: 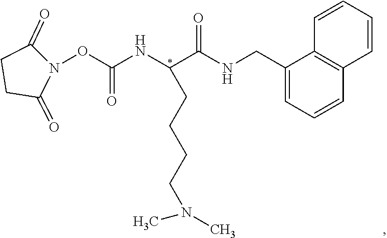 [SMILES: CN(C)CCCCC(NC(=O)ON1C(=O)CCC1=O)C(=O)NCc1cccc2ccccc12]
wherein the carbon atom marked with an asterisk is the at least one chiral carbon atom.

11. The chiral derivatization reagent of claim 4, wherein the chiral derivatization reagent having Formula (I) has the structure of: 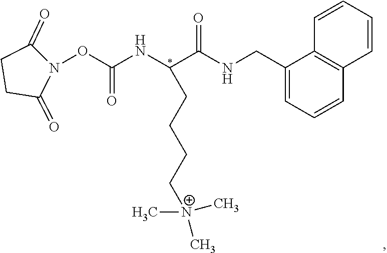 [SMILES: C[N+](C)(C)CCCCC(NC(=O)ON1C(=O)CCC1=O)C(=O)NCc1cccc2ccccc12]
wherein the carbon atom marked with an asterisk is the at least one chiral carbon atom.

12. The chiral derivatization reagent of claim 4, wherein the chiral derivatization reagent having Formula (II) has a structure selected from: 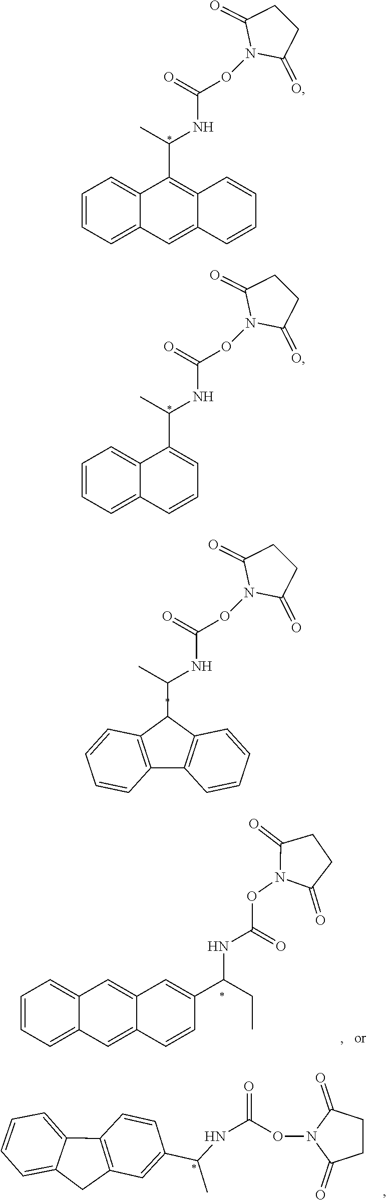
wherein the carbon atom marked with an asterisk is the at least one chiral carbon atom.

13. The chiral derivatization reagent of claim 4, wherein the chiral derivatization reagent having Formula (III) has the structure of: 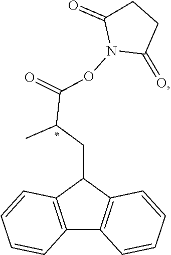 [SMILES: CC(CC1c2ccccc2-c2ccccc21)C(=O)ON1C(=O)CCC1=O]
wherein the carbon atom marked with an asterisk is the at least one chiral carbon atom.

14. The chiral derivatization reagent of claim 4, wherein the mass of the derivatization reagent has a molecular weight of about 150 to about 600 atomic mass units.

15. The chiral derivatization reagent of claim 4 for separating amino group-containing enantiomeric isomers in a sample.

16. A method of separating amino group-containing enantiomeric isomers, comprising using the chiral derivatization reagent of claim 4.

17. A method for analyzing an amino group-containing enantiomeric isomers in a sample comprising:
a) reacting the sample comprising the amino group-containing enantiomeric isomers with a chiral derivatization reagent of claim 4;
b) allowing each amino group-containing enantiomeric isomers to react with the chiral derivatization reagent of claim 4 to produce a mixture of diastereomers comprising the derivatization reagent and a specific amino group-containing enantiomeric isomer;
c) loading the mixture of diastereomers onto a chromatographic separation device;
d) eluting the mixture of diastereomers from the chromatographic separation device; and analyzing the eluent for the presence of a specific diasteromer comprising the specific amino group-containing enantiomeric isomer using a mass spectroscopy.

18. The method of claim 17, wherein the chromatographic separation device is a device selected from the group consisting of a chromatographic column, a thin layer plate, a filtration membrane, a microfluidic separation device, a sample cleanup device, a solid support, a solid phase extraction device, a microchip separation device, and a microtiter plate.

19. The method of claim 17, wherein the chromatographic separation device is a liquid chromatography column comprising an achiral stationary phase.

20-21. (canceled)

22. The method of claim 17, wherein the mass spectroscopy is real-time online mass spectroscopy.

23. (canceled)

24. The method of claim 17, wherein the sample is a biological sample comprising a bodily fluid, and the bodily fluid is selected from the group consisting of saliva, sweat, urine, blood, serum, plasma, spinal fluid, and combinations thereof.

25. (canceled)

26. The method of claim 17, wherein the amino group-containing enantiomer is an amino acid or a biogenic amine.

27. A chiral derivatization reagent comprising at least one chiral carbon atom, the chiral derivatization reagent having a formula of: 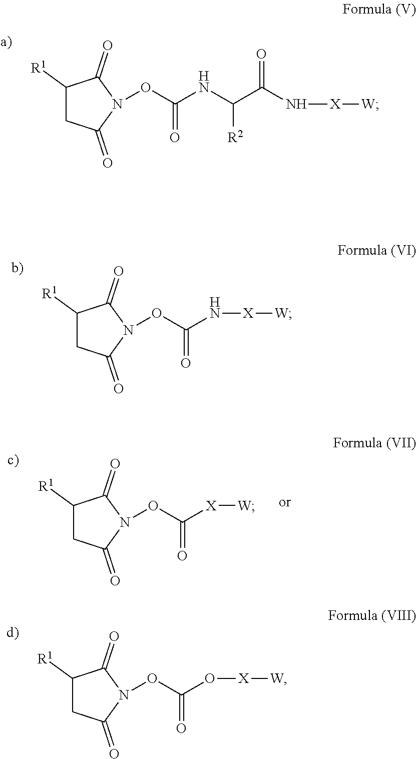
wherein
R¹ is an optional sulfo group, R² is an natural or non-natural amino acid side chain or derivative thereof, X is a linker group comprising linear or branched substituted alkyl chain, linear or branched unsubstituted alkyl chain, linear or branched substituted heteroalkyl chain, or linear or branched unsubstituted heteroalkyl chain, W is —[Y—Z] or —[Z—Y], wherein—is the attachment point to X, Y is at least one chromophore moiety or at least one fluorophore moiety, and Z is strongly basic moiety having a pKa value greater than 8.

28-31. (canceled)
